(19) United States
(12) Patent Application Publication
(10) Pub. No.: US 2011/0256087 A1
(43) Pub. Date: Oct. 20, 2011

(54) COSMETIC COMPOSITION CONTAINING A LOCUST BEAN GUM HYDROLYSATE

(75) Inventors: Bernard Fabre, Belberaud (FR); Christel Fiorini-Puybaret, L'Union (FR)

(73) Assignee: PIERRE FABRE DERMO-COSMETIQUE, Boulogne-billancourt (FR)

(21) Appl. No.: 13/141,286

(22) PCT Filed: Dec. 23, 2009
(86) PCT No.: PCT/EP2009/067831

§ 371 Date: Jun. 29, 2011

(30) Foreign Application Priority Data
Dec. 23, 2008 (FR) 0859025

(51) Int. Cl. A61K 8/97; A61Q 5/06; A61K 36/48

(52) U.S. Cl. 424/74; 424/195.18

(57) ABSTRACT

The invention relates to a locust bean gum hydrolysate, to a method for preparing same, and to the use thereof in hair cosmetics.

The invention relates to the preparation of a locust bean gum hydrolysate and to the use thereof in a cosmetic hair composition.

The locust bean is the fruit of the carob tree, Ceratonia siliqua L., a dioecious plant of the fabaceae family, originating from Mediterranean regions (North Africa, Near East, Southern Europe) and the Canary Islands. The locust bean gum, also called carob bean flour, is obtained by grinding the endosperm of carob tree beans, contained in pods. The seeds of the locust bean are brown, of flattened ovoid shape, biconvex and very hard. They are separated from each other by pulpy septa. There are 15 to 20 per pod. The pods are also called “locust beans”. They are hanging, from 10 to 30 cm long and 1.5 to 3 cm wide, firstly of green colour, then dark brown in maturity.

The production of the locust bean gum thus requires an optimal separation of the seeds from the pod on the one hand, then the endosperm from the other parts of the seed, the endocarp and the germ, on the other hand. Locust bean gum consists essentially of a polysaccharide of galactomannan type, the molecular weight of which ranges from 50,000 to several million. The locust bean gum forms with water a colloid, which reaches its optimal solubilisation when the water is heated to a temperature of 80 to 90° C. The solutions obtained are slightly cloudy and have a very high viscosity.

This gum is particularly known in the food industry as a thickener and gelifier in soups, sauces, creams, ice creams, etc. The pharmaceuticals industry also uses it for these same properties.

Due to its high gelifying and viscosifying power, it is very difficult to use locust bean gum in a fluid cosmetic formula such as a shampoo. In addition, due to its high molecular weight, locust bean gum applied to hair would have an inaesthetic and undesirable weighting effect in cosmetics. Despite these negative effects, and due to its chemical structure and its affinity for hair, the locust bean gum has interesting properties such as its coating and protective effect towards hair.

There exists a whole range of products added to the shampoo or to the conditioner that make it possible to condition the hair and provide it with new characteristics that are not found in a basic shampoo, whether a protecting effect, a stylising effect, or the addition of a particular colour, for example. Thus, the use of polysaccharide derivatives in hair products is known. Their physical-chemical characteristics (viscosity, solubility, colour, etc.) enable them to be incorporated more or less easily in said hair compositions. Products with long polysaccharide chains of natural extracts are, as such, difficultly soluble in cosmetic compositions and generally have to undergo human intervention by chemical means, for example in order to render them usable.

The applicant has thus sought to modify locust bean gum in order to overcome the aforementioned drawbacks, such as the weighting effect and to maintain the coating and protective benefit of said gum towards hair.

Thus, the applicant has prepared in a surprising manner a locust bean gum hydrolysate having new physical-chemical properties enabling it to be incorporated in a shampoo. The applicant has then noted a recognised and unexpected volume enhancing effect on hair of said hydrolysate.

The object of the invention is thus a locust bean gum hydrolysate having a reducing sugars content between 10% and 60%, advantageously between 25% and 45% by weight compared to the weight of dry matter of said hydrolysate.

A hydrolysate is a product stemming from a hydrolysis reaction of a substrate, here locust bean gum. A hydrolysis is a decomposition of a body by fixation of H⁺ and OH⁻ ions stemming from the dissociation of water. Thus, the presence of water is necessary for the hydrolysis to take place. The substrate may be dissolved in pure water or in a mixture of water and one or more other solvents.

A reducing sugar is a sugar comprising a terminal aldehyde function that acts as an electron donor in an oxidation-reduction reaction. Historically, this term stems from the discovery of Fehling in the 19th century that proved that certain sugars reacted with cupric ions to transform them into cuprous ions. Such an assay makes it possible to quantify the terminal functions of a sugar. The assay of the reducing sugars makes it possible to measure the efficiency of hydrolysis and to assay the oligomers generated.

The best results of the volume enhancing effect have been observed when the reducing sugars content of the locust bean gum hydrolysate lies in a range between 10% and 60%, and even better in a range from 25 to 45% by weight compared to the weight of dry matter of said hydrolysate.

Locust bean gum is essentially constituted of a characteristic polygalactomannan, comprising a fundamental long chain composed uniquely of mannoses bonded by β 1-4-glycosidic bonds. This chain bears branches constituted of a single unit of galactose bonded to the chain of mannoses by a-glycosidic bonds. This polysaccharide is constituted of mannose and galactose in a natural proportion situated approximately at 4:1. It may be assumed that the hydrolysis is thus going to fall essentially on this polysaccharide, but also on the compounds or traces of compounds accompanying this polysaccharide on the locust bean gum.

The locust bean gum hydrolysate according to the invention may be a chemical and/or physical and/or biological hydrolysate, in other words stem from chemical and/or physical and/or biological methods.

In a particular embodiment, the locust bean gum hydrolysate according to the invention is an enzymatic hydrolysate advantageously obtained by a mannose type enzyme.

Enzymes of mannase type are hydrolases, more specifically, osidases. They are thus going to be capable of hydrolysing the osidic bond in the “mannose-mannose” unit situated in polysaccharide fragments or any other molecule comprising this unit. Thus, the action of mannase on the locust bean gum is going to produce polysaccharides of reduced sizes compared to the initial galactomannan.

The locust bean gum hydrolysate may be in the form of a dry extract, in particular in the form of a powder or any solid, or a fluid or viscous liquid extract.

Dry extract of locust bean gum hydrolysate is taken to mean the set of all the substances that do not volatilise and are from the hydrolysed locust bean gum.

Liquid extract of locust bean gum hydrolysate is taken to mean the set of all the substances from the hydrolysis of the locust bean gum in inorganic or organic solution.

Another object of the invention is a method of preparing a locust bean gum hydrolysate presenting the following steps:

a) hydrolysis of the locust bean gum. The hydrolysis takes place in water.

b) interruption of the hydrolysis when the reducing sugars content is between 10% and 60%, advantageously between 25% and 45% by weight in relation to the weight of dry matter of said hydrolysate.

c) solid/liquid separation of the mixture obtained at step b) to obtain a locust bean gum hydrolysate.

Advantageously, step c) of separation is carried out by filtration and/or centrifugation.

The method can take place at step a) by a hydrolysis by physical and/or chemical and/or biological route.

Several techniques can produce a locust bean gum hydrolysate. A source of water is obligatory in order to obtain a hydrolysate. For example, a chemical hydrolysis may take place by means of a strong acid.

A physical hydrolysis may take place by ultrafiltration.

A biochemical hydrolysis may take place with an enzyme.

A biological hydrolysis may take place with a living organism such as a micro-organism.

The final result of all these methods is a locust bean gum hydrolysate that may be characterised by its reducing sugars content.

The measurement of the level of reducing sugars may be done by any conventional manner well known to those skilled in the art. For example it may be carried out by an assay by 3,5-dinitrosalicylic acid (DNS), also still known as 2-hydroxy-3,5-dinitrobenzoic acid. The DNS is reduced into 3-amino-5-nitrosalicylic acid by the reducing sugar according to the following reaction:

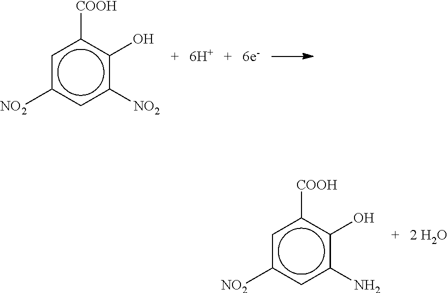

The sugar is for its part oxidised into various oxidation products:

Sugar->Oxidation products+n.e⁻

3-amino-5-nitrosalicylic acid is a red compound. This reaction is not stoichiometric: there is no oxidation-reduction balance. The intensity of the red coloration is proportional to the concentration of the terminal aldehyde function, if constant physical-chemical conditions are employed. The calibration lines do not always go through the origin.

The interruption of the hydrolysis of locust bean gum is specific to the chosen hydrolysis technique: stoppage of the physical phenomenon used, stoppage of the chemical phenomenon by neutralisation of the pH, denaturation of the enzyme brought into play by rise in temperature or addition of alcohol, sterilisation of the hydrolysate.

The locust bean gum hydrolysate may then be filtered on a membrane or a frit.

The hydrolysate may also be centrifuged in order to recover a clear supernatant, free of particles.

The method may further comprise a subsequent step d) of concentration of the hydrolysate obtained at step c).

This is carried out by any technique of evaporation of the water used during the hydrolysis. Thus, it can be done by placing under more or less marked vacuum with, if appropriate, a rise in the temperature of the hydrolysate or by any other method well known to those skilled in the art.

The method may also further comprise a step of adding a microbial preservative and/or adding solvent(s) among glycerine, propylene glycol, butylene glycol, ethanol or any solvent enabling a microbiological property. This step can take place before concentration or after partial concentration. The quantities of solvent to add may vary according to their anti-microbial properties.

In the case of a microbial preservative added directly to the aqueous hydrolysis solution, said microbial preservative may be chosen from the following list: cetrimonium chloride, ethanol, capryl glycol, glycerine, for example. This addition of solvents or preservatives can take place at any step described. The extracts thereby obtained have lost their colloidal property and may be miscible in water and/or diluted with water.

The hydrolysate may also be in solid form, such as a dry extract. The method then further comprises a subsequent step e) of drying of the hydrolysate to obtain a dry extract of locust bean gum hydrolysate. Firstly, the hydrolysate is concentrated then dried in an oven under vacuum for example with a temperature between 40 and 90° C., or by nebulisation, by lyophilisation or any other method. The extracts thereby obtained have lost their colloidal property and are very soluble in water.

According to a preferred embodiment of the invention, step a) of the process for preparing a locust bean gum hydrolysate according to the invention is carried out by an enzymatic hydrolysis, advantageously by an enzyme of mannase type.

Advantageously, within the scope of said preferred embodiment, step a) of the method of manufacture consists in placing in contact an aqueous solution of the locust bean gum under suitable conditions of temperature and pH with an enzyme of mannase type. Thus, the enzyme is solubilised in water initially under constant stirring. The water is heated beforehand to a temperature that can vary between 30° C. and 70° C., preferentially between 40° C. and 50° C. The proportions of water compared to the enzyme vary in a water/enzyme ratio (weight/weight) from 10/1 to 1000/1 and preferentially from 20/1 to 200/1. The locust bean gum is then added to the solution in a water/gum ratio (weight/weight) that can vary from 5/1 to 20/1, preferentially from 7/1 to 12/1. Thus, the method of preparing an enzymatic hydrolysate of locust bean gum may have a reaction ratio between the locust bean gum and the enzyme of mannase type during step a) of between 1/2 and 200/1 by dry weight.

The pH may then be adjusted by addition of acid (HCl, H₂SO₄, CH₃COOH for example) or base (KOH, NaOH for example) so as to attain values situated between 2 to 7 and preferentially between 4 and 5.

The stirring and the temperature are maintained throughout the enzymatic hydrolysis for 30 minutes to 10 h and preferentially between 1h30 and 3h. When the reducing sugars content reaches the desired value, the hydrolysis may be interrupted. Within the scope of this preferred embodiment, the method bringing into play an enzymatic hydrolysis at step a) is preferably interrupted at step b) by a rise in temperature between 70 and 100° C., advantageously between 95 and 100° C., for a duration varying from 5 minutes to 30 minutes, or by the addition of an alcohol.

Another object of the invention also relates to a cosmetic composition comprising, as active ingredient, a locust bean gum hydrolysate according to the invention.

Thus, the cosmetic composition of the invention comprises, as active ingredient, a locust bean gum hydrolysate with a reducing sugars content between 10% and 60% by weight compared to the weight of dry matter of said hydrolysate, preferentially with a reducing sugars content between 25% and 45% by weight compared to the weight of dry matter of said hydrolysate.

In an advantageous manner, the cosmetic composition of the invention comprises, as active ingredient, a locust bean gum hydrolysate of the invention, enzymatic, preferentially obtained by an enzyme of mannase type.

The locust bean gum hydrolysate of the invention may be in the cosmetic composition of the invention in the form of a dry extract or a liquid extract.

Preferentially, the cosmetic composition according to the present invention comprises a quantity of dry extract of locust bean gum hydrolysate according to the invention of between 0.1 g and 2.0 g, and preferentially between 0.1 g and 1.0 g for 100 g of said composition and in a particularly preferred manner around 0.5 g for 100 g of cosmetic composition.

The cosmetic composition according to the present invention may advantageously be in all forms normally used in the cosmetics field for topical application. Thus and preferentially, it may be in the form of a shampoo, a gel, a lotion, a foam, a spray, a dispersion, a serum, a mask, a body lotion, a volumising balm, or a cream for example.

In a preferential manner, the cosmetic composition according to the invention is a hair composition.

Hair composition is taken to mean an aqueous and/or organic composition intended for the washing, the colouring, the care or the styling of hair. Advantageously, it fixes the hair style and/or provides a beneficial effect to the cosmetic state of the hair and/or provides sheen to the hair, and/or makes it possible to maintain or shape the hair style.

The compositions conforming to the invention may be used for the washing or the treatment of keratinic materials such as hair, eyelashes, the scalp and more particularly hair.

The cosmetic composition according to the invention comprises, moreover, usual cosmetically compatible excipients.

The usual excipients compatible with the cosmetic hair composition of the invention may be any excipient known to those skilled in the art so as to obtain a cosmetic composition for topical application in the forms as described above.

The cosmetic composition according to the invention may in particular contain additives and formulation aids, such as surfactants of emulsifying, cleaning, foaming type, etc., complexing agents, thickeners, gelifiers, stabilisers, preservatives including antimicrobial preservatives and antioxidants, conditioners, acidifiers, alkalizers, emollients, solvents, colorants, fragrances.

The hair composition of the invention may comprise other compounds useful for the conditioning of hair, such as colouring or sheen agents.

Another object of the invention is the use of a locust bean gum hydrolysate according to the invention as cosmetic agent.

Thus, a locust bean gum hydrolysate characterised by a reducing sugars content between 10% and 60% by weight compared to the weight of dry matter of said hydrolysate, preferentially by a reducing sugars content between 25% and 45% by weight compared to the weight of dry matter of said hydrolysate, is used as cosmetic agent.

In an advantageous manner, the locust bean gum hydrolysate of the invention used as cosmetic agent is an enzymatic hydrolysate, preferentially obtained by an enzyme of mannase type.

Moreover, a locust bean gum hydrolysate of the invention may be used as cosmetic agent in the form of a dry extract or a liquid extract.

Advantageously, the locust bean gum hydrolysate of the present invention is used as cosmetic agent in a cosmetic composition, preferably a hair composition.

Another object of the invention is the use of a hair composition according to the invention to give a volume enhancing effect to hair.

Another object of the invention is the use of a hair composition according to the invention to coat hair while protecting it, advantageously for an application on thin hair and/or hair without volume.

The hair composition according to the invention is intended to create volume from the roots to the tips of the hair. The hair is then perfectly shapely for a long lasting volume. The texture of the hair is thereby improved.

The following examples illustrate the invention without restricting the scope thereof.

### EXAMPLE 1

### Conditions of Enzymatic Hydrolysis of Locust Bean Gum by an Enzyme of Mannase Type

1000 litres of water are heated in a reactor to a temperature of 45° C. while stirring. 10 kg of mannase are added. After dissolution, 100 kg of locust bean flour are added. The pH is adjusted to 4.5.

The temperature and the stirring are maintained for 3 hours, then the temperature is raised to 95° C. for 10 minutes. After cooling, a solid/liquid separation on filter is carried out. The solution collected is concentrated then dried. 80 kg of hydrolysed locust bean gum are thereby obtained, the reducing sugars content of which is around 35% (weight/weight).

### EXAMPLE 2

### Conditions of Enzymatic Hydrolysis of Locust Bean Gum by an Enzyme of Mannase Type

100 litres of water are heated in a reactor to a temperature of 50° C. while stirring. 0.5 kg of mannase is added, then 20 kg of locust bean gum. The pH is adjusted to 4. The hydrolysis is maintained for 2 hours, then the temperature is raised to 95° C. for 15 minutes. After cooling, the solution is filtered then an equal volume of propylene glycol is added to the filtrate. The assays of reducing sugars indicate a value compared to dry matter of around 20% (weight/weight).

### EXAMPLE 3

### Study of the Efficiency of the Locust Bean Gum Hydrolysate in Hair Cosmetic

A study of the efficiency in hair cosmetic made it possible to evaluate the activity of a 1% aqueous solution of the hydrolysed locust bean gum obtained by the method described in example 1 on the volume of locks of hair in comparison with an untreated control. To do this, the locks are washed beforehand in a standardised manner then dried, also in a standardised manner. These locks are then immersed in the aqueous solution of hydrolysed locust bean gum or in water for 3 minutes. Said locks are then dried for 18 hours on a “birdcage” shaped mould. The activity on the volume of the hair is measured by image analysis. To do this, the locks are placed opposite a light source and undergo a rotation of 180° . During this rotation, the volume of the locks in movement is projected onto a screen and their surface is measured. The greater the surface area, the greater the volume enhancing effect of the products tested. Analysis of the results demonstrates a significant difference in the volume of the locks of +11% between the 2 groups in favour of the group treated by the hydrolysed locust bean gum, and thus justifies its use in a volume enhancing shampoo.

### EXAMPLE 4 Composition

### Volume Enhancing Shampoo

| Locust bean gum hydrolysate dry extract | 0.3 to 1.0 | g |
|---|---|---|
| Ammonium lauryl sulphate | 1.5 | g |
| Sodium laureth sulphate | 10.0 | g |
| Cocoamphodiacetate | 10.0 | g |
| Ceteareth-60 myristyl glycol | 0.75 | g |
| Disodium EDTA | 0.2 | g |
| Hydroxypropyl guar | 0.25 | g |
| Capryl glycol | 1.0 | g |
| Cetrimonium chloride | 2.0 | g |
| Monohydrated citric acid | sqf pH = 5 | |
| Colorant(s) | sq. | |
| Fragrance | sq. | |
| Purified water | sqf 100 | g |

### EXAMPLE 5

### Composition: Sprayable Gel Without Volume Enhancing Rinsing

| Locust bean gum hydrolysate dry extract | 0.1 to 0.7 | g |
|---|---|---|
| Xanthane gum | 0.3 | g |
| Vinylpyrrolidone/vinylacrylate copolymer | 1.0 | g |
| Dimethicone copolyol | 0.3 | g |
| Castor oil | 0.3 | g |
| Denatured ethyl alcohol | 6.0 | g |
| Disodium EDTA | 0.2 | g |
| Fragrance | sq | |
| Preservative(s) | sq | |
| Sodium hydroxide | sqf pH = 5.5-6.5 | |
| Purified water | sqf 100 | g |

### EXAMPLE 6

### Composition: Volume Enhancing Foam

| Locust bean gum hydrolysate dry extract | 0.1 to 0.7 | g |
|---|---|---|
| Vinylpyrrolidone/vinylacrylate copolymer | 1.0 | g |
| Cetrimonium chloride | 1.0 | g |
| Quaternized polyvinyl pyrrolidone-acrylamide | 3.0 | g |
| Glycerine | 0.5 | g |
| Panthenol | 0.2 | g |
| Ceteareth-20 | 0.2 | g |
| Hydroxyethylcellulose | 0.1 | g |
| Fragrance | sq | |
| Monohydrated citric acid | sqf pH = 5-6 | |
| Purified water | sqf 100 | g |

1. Cosmetic composition comprising, as active ingredient, a locust bean gum hydrolysate characterised by a reducing sugars content between 10% and 60% by weight compared to the weight of dry matter of said hydrolysate.

2. Cosmetic composition according to claim 1, characterised in that the locust bean gum hydrolysate comprises a reducing sugars content between 25% and 45% by weight compared to the weight of dry matter of said hydrolysate.

3. Cosmetic composition according to any of claims 1 or 2, characterised in that the locust bean gum hydrolysate is an enzymatic hydrolysate.

4. Cosmetic composition according to claim 3, characterised in that it is an enzymatic hydrolysate obtained by an enzyme of mannase type.

5. Cosmetic composition according to any of the preceding claims, characterised in that the locust bean gum hydrolysate is a hydrolysate being in the form of a dry extract or a liquid extract.

6. Cosmetic composition according to any of the preceding claims comprising a quantity of locust bean gum hydrolysate between around 0.1 g and 2.0 g for 100 g of said composition.

7. Cosmetic composition according to any of the preceding claims, being in the form of a shampoo, a gel, a lotion, a foam, a spray, a dispersion, a serum, a mask, a body lotion, a volumising balm or a cream.

8. Hair composition according to any of the preceding claims.

9. Use of a locust bean gum hydrolysate characterised by a reducing sugars content between 10% and 60% by weight compared to the weight of dry matter of said hydrolysate as cosmetic agent.

10. Use of a locust bean gum hydrolysate as cosmetic agent according to claim 9, characterised by a reducing sugars content between 25% and 45% by weight compared to the weight of dry matter of said hydrolysate.

11. Use of a locust bean gum hydrolysate as cosmetic agent according to any of claim 9 or 10, characterised in that the locust bean gum hydrolysate is an enzymatic hydrolysate.

12. Use of a locust bean gum hydrolysate as cosmetic agent according to claim 11, characterised in that it is an enzymatic hydrolysate obtained by an enzyme of mannase type.

13. Use of a locust bean gum hydrolysate as cosmetic agent according to any of claims 9 to 12, characterised in that the locust bean gum hydrolysate is a hydrolysate being in the form of a dry extract or a liquid extract.

14. Use of a locust bean gum hydrolysate as cosmetic agent according to any of claims 9 to 13 in a cosmetic composition, preferably a hair composition.

15. Use of a hair composition according to claim 8 to give a volume enhancing effect to hair.

16. Use of a hair composition according to claim 8 to coat hair while protecting it, advantageously for application on thin hair and/or hair without volume.
